(19) United States
(12) Patent Application Publication
(10) Pub. No.: US 2016/0331698 A1
(43) Pub. Date: Nov. 17, 2016

(54) FULLERENE COMPOUNDS AS ANTIOXIDANT THERAPY

(71) Applicants: Kiminobu SUGAYA, Orlando, FL (US); Alexander TORRES, Oviedo, FL (US)

(72) Inventors: Kiminobu SUGAYA, Orlando, FL (US); Alexander TORRES, Oviedo, FL (US)

(73) Assignee: University of Central Florida Research Foundation, Inc., Orlando, FL (US)

(21) Appl. No.: 15/111,331

(22) PCT Filed: Jan. 13, 2015
(86) PCT No.: PCT/US15/11268

§ 371 Date: Jul. 13, 2016

Related U.S. Application Data
(60) Provisional application No. 61/926,883, filed on Jan. 13, 2014.

(51) Int. Cl. A61K 31/047; A61Q 19/08; A61K 8/34

(52) U.S. Cl. CPC A61K 31/047; A61K 8/345; A61Q 19/08; A61K 2800/74; A61K 2800/592

(57) ABSTRACT

Disclosed herein are small-gap fullerene compositions and methods of using for therapeutic or cosmetic purposes. Specifically exemplified is the use of derivatized SGFs so as to be water-soluble. The derivatized SGFs include polyhydroxylated and polycarboxylated SGFs.

### BACKGROUND

Alzheimer's Disease is currently an incurable disease that is growing over time. A current treatment-aim is to inhibit the neurotoxic effects of a protein compound found in Alzheimer's disease. The neurotoxic protein in the brain of patients with Alzheimer's disease, called Amyloid-beta (Abeta) results in the cells' production of free radicals. In Alzheimer's there are low levels of natural antioxidants that can potentially mitigate the effects of Abeta. The imbalance between free radicals and antioxidants in the brain is called oxidative stress.

### DETAILED DESCRIPTION

In certain embodiments, disclosed herein are fullerene containing antioxidant compositions such as those comprising a polyhydroxylated small-gap fullerene (PSGF) compound, a basic, soluble carbon nanotube derivative, and methods of using same. It is disclosed herein that PSGF compounds, and in particular PSGF compounds of a size in the range of C60-C400 demonstrate remarkable antioxidant activity. In particular, a PSGF composition comprising C60-C400 PSGF demonstrated antioxidant activity against free radicals produced by NT-2 (neuron-like) cells in response to Abeta (e.g. Abeta42) exposure.

Fullerenes with small energy differences between filled and unfilled electronic states of the individual fullerene molecules (“small gap fullerenes”) are especially susceptible to such spontaneous polymerization, and are therefore not recovered by solvent extraction. The percentage of isomers of a certain cage size which are “small gap” increase dramatically as the cage size increases; thus, solvent-extracted fullerenes have few giant fullerenes, while small gap fullerenes (SGFs) are rich in giant fullerenes larger than C₈₄. Despite their relative abundance, SGFs are usually therefore discarded along with the non-fullerenic carbon matrix. SGFs which have been modified or derivatized to increase water solubility, such as by hydroxylation or carboxylation may be used in accordance with the teachings herein and are included within the term SGF unless otherwise indicated.

An example of a PSGF compound useful with the embodiments herein includes polyhydroxy small gap fullerenes commercially available from Sigma-Aldrich, St. Louis, Mo. (Sigma Product Number 707481 a representative structural formula is provided below:

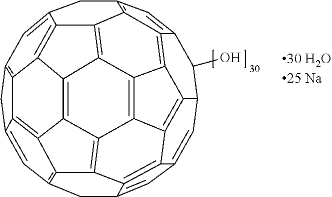

Raebiger et al., Carbon 2011, 49:37-46; Zemanova et al., Journal of Materials Science and Engineering, 2012, 2:86-97; Wang et al., J. Med. Chem. 1999, 42 (22): 4614-4620; S Nakamura and T Mashino 2009 J. Phys.: Conf. Ser. 159 012003; Foley et al. Biochemical and Biophysical Research Communications, 2002, 294:116-119; and Liu et al., ACS Appl. Mater. Interfaces, 2013 5(21):11101-11107 are cited and incorporated herein by reference in its entirety for background information concerning fullerene compounds, including methods of making and derivatizing (e.g. hydroxylation or carboxylation) fullerene compounds that may be used in accordance with the teachings herein. It will be recognized that the derivitization techniques provided in the foregoing papers can be applied to SGF fullerenes.

The term “fullerene” is used generally herein to refer to any closed cage carbon compound containing both six- and five-member carbon rings independent of size and is intended to include the abundant lower molecular weight C60 and C70 fullerenes, larger known fullerenes including C₇₆, C₇₈, C₈₄ and higher molecular weight fullerenes C_{2N} where N is 50 or more (giant fullerenes) which may be nested and/or multi-concentric fullerenes. The term is intended to include “solvent extractable fullerenes” as that term is understood in the art (generally including the lower molecular weight fullerenes that are soluble in toluene or xylene) and to include higher molecular weight fullerenes that cannot be extracted, including giant fullerenes which can be at least as large as C400. Additional classes of fullerenes include, among others specifically noted herein, endohedral fullerenes containing one or more elements, particularly one or more metal elements, and heterofullerenes in which one or more carbons of the fullerene cage are substituted with a non-carbon element, such as B or N. The term fullerenic material is used generally to refer to a material that contains a mixture of fullerenes or a mixture of one or more fullerenes with non-fullerenes, e.g., amorphous carbonaceous materials that may for example be formed during fullerene synthesis by any known method and includes raw or crude preparations of fullerenes, such as combustion soot as well as raw or crude preparations of fullerenes that have been at least partially purified, for example, by extraction and/or sublimation.

The term “SGF composition” pertains to a composition that comprises SGFs optionally combined with a pharmaceutically acceptable carrier. The SGFs of the composition may be water soluble or insoluble, but are typically water soluble (e.g. derivatized to be water soluble). The SGF composition may include giant fullerenes having a molecular weight of greater than C₈₄, and typically are C₁₀₀ or higher. The SGF composition typically has more solvent inextractable fullerenes than solvent extractible fullerenes. In a certain embodiment, the SGF composition has a peak distribution of SGFs of between about C₁₀₀ to C₂₀₀. In a specific embodiment, the peak distribution is about C₁₂₀ In alternative embodiment, the SGF composition comprises SGFs of a size that is large enough to inhibit uptake into cells and/or to cross the blood-brain barrier.

The term “PSGF composition” refers to an SGF composition having at least about fifty percent of the SGFs being polyhydroxylated. In specific embodiments, the PSGF composition has at least about 55, 60, 65, 70, 75, 80, 85, 90, or 95 percent of the SGFs being polyhydroxylated.

Also disclosed are methods of ameliorating, preventing, delaying the onset or improving an unwanted condition, disease or symptom of a patient associated with oxidative stress. In particular, the method involves the administration of a SGF to a subject in need thereof, such as a subject under oxidative stress in response to amyloid beta. The condition may include neurodegenerative disorders, cardiovascular disease, as well as aging of the skin. Cosmetic uses typically involve administration of a PSGF composition topically to the skin of a subject.

As used herein the term “oxidative stress” pertains to conditions where an imbalance occurs between the systemic manifestation of reactive oxygen species or free radicals and a biological system's ability to readily detoxify the reactive species or to repair the resulting damage.

As used herein and in the appended claims, the singular forms “a”, “an”, and “the” include plural reference unless the context clearly dictates otherwise. Thus, for example, reference to an “antioxidant” is a reference to one or more antioxidants and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic to a patient whereby the therapeutic positively impacts the tissue to which it is targeted. The compounds described herein can be administered either alone or in combination (concurrently or serially) with other pharmaceuticals. For example, the compounds can be administered in combination with other antioxidants or agents known to treat the target condition. In some embodiments, the compounds described herein can also be administered in combination with (i.e., as a combined formulation or as separate formulations) with antibiotics.

The terms “animal,” “patient,” or “subject” are used interchangeably, and include, but are not limited to, humans and non-human vertebrates such as wild, domestic and farm animals. Typically, the term refers to humans.

By “pharmaceutically acceptable”, it is meant the carrier, diluent or excipient must be compatible with the other ingredients of the formulation and not deleterious to the recipient thereof.

As used herein, the term “therapeutic” means an agent utilized to discourage, combat, ameliorate, prevent or improve an unwanted condition, disease or symptom of a patient.

A “therapeutically effective amount” or “effective amount” of a composition is a predetermined amount calculated to achieve the desired effect, i.e., to treat or prevent an unwanted condition, disease or symptom of a patient. In a specific example, a therapeutically effective amount is one that reduces the adverse cellular effects of oxidants such as reactive oxygen species (ROS) or free radicals, including those associated with Abeta. The activity contemplated by the present methods includes both therapeutic and/or prophylactic treatment, as appropriate. The specific dose of the compounds or the compounds administered according to this invention to obtain therapeutic and/or prophylactic effects will, of course, be determined by the particular circumstances surrounding the case, including, for example, the compounds administered, the route of administration, and the condition being treated. The effective amount administered may be determined by a physician in the light of the relevant circumstances including the condition to be treated, the choice of compounds to be administered, and the chosen route of administration. A therapeutically effective amount of the compound/compound of this invention is typically an amount such that when it is administered in a physiologically tolerable excipient composition, it is sufficient to achieve an effective systemic concentration or local concentration in the target tissue.

As used herein, the term “treat”, “treating” and “treatment” refers to an improvement in the disease condition. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease.

As used herein, the term “prevent”, or “prevention” refers to delaying the onset of a disease condition in a subject or reducing the risk of occurrence of the disease condition. Administering an SGF composition to a patient who exhibits increased oxidative stress markers, but who has not exhibited symptoms of a disease condition is an example of preventing an oxidative stress induced disease such as a neurodegenerative disease.

The terms “prophylactically effective (or efficacious) amount” and similar descriptions such as “an amount efficacious for prevention” are intended to mean that amount of a pharmaceutical drug that will prevent or reduce the risk of occurrence of the biological or medical event that is sought to be prevented in a tissue, a system, animal or human. The dosage regimen utilizing an SGF compound is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the prophylactically effective dosage amount needed to prevent the condition. It is understood that a specific daily dosage amount can simultaneously be both a therapeutically effective amount, e.g., for treatment of hypertension, and a prophylactically effective amount, e.g., for prevention of myocardial infarction.

Markers for oxidative stress include but are not limited to isoprostanes (IsoPs), malondialdehyde (MDA), nitrotyrosine, S-glutathionylation, myeloperoxidase (MPO), oxidized low-density lipoprotein (OxLDL), ROS-induced changes in gene expression, or net antioxidant capacity, advanced glycation end products (AGEs), 8-hydroxyguanosine (8OHG), 4-hydroxy-trans-2-nonenal (HNE), 4-oxo-trans-2-nonenal (4-ONE), acrolein, and 4-oxo-trans-2-hexenal. See Ho et al. Redox Biology, 2013, 1:483-491. Moreira et al., Methods Mol Biol, 2010, 610:419-34; Jomova et al., Mol cell Biochem 2010 345:91-104. These markers can be tested to determine if they elevated in a subject.

Generally speaking, the term “tissue” refers to any aggregation of similarly specialized cells which are united in the performance of a particular function.

Alzheimer's disease (AD) is a progressive neurodegenerative disorder characterized by cognitive decline, irreversible memory loss, disorientation, and language impairment. AD affects 10% of the population aged greater than 65 and at least 50% of the population aged greater than 85 years. AD has been reported in patients as young as 40-50 years of age, but because the presence of the disease is difficult to detect without histopathological examination of brain tissue, the time of onset in living subjects is unknown. Several etiological factors have been implicated in the pathogenesis of Alzheimer's disease. These factors lead to the activation of a cascade process that brings about neuronal death and serious decline in cognitive function. These bed-ridden patients ultimately succumb to death due to inter-current infections related to aspiration, decubitus and stagnation of urine. In one embodiment, PSGF is administered to a subject exhibiting symptoms of AD, including for example decrease cognitive function.

AD involves, biochemically, a pathological cleavage of amyloid precursor protein (APP). APP, in normal circumstances, is cleaved by a- and γ-secretases and takes part in axonal transport, synapse formation and synaptic repair in the CNS. The abnormal, sequential processing by the beta-site amyloid precursor protein-cleaving enzymes (BACE) and γ-secretase results in amyloid-β, which is highly neurotoxic. Molecules of amyloid-β, especially the amyloid-i_42 type, are prone to aggregation and accumulation in the cell membrane forming insoluble aggregates called “rafts”. Subsequently, these impair membrane conductivity, Ca²⁺ fluxes, control of the formation of reactive oxygen species (ROS), τ-protein assembly, axonal transport, and the polarity of mitochondrial membrane. Ultimately, the pathologic cascade leads to Ca²⁺ toxicity, activation of apoptotic processes, inflammation and neuronal death. This vicious cycle can be initiated by a wide array of triggering mechanisms that can be traced back to genetic or environmental factors. Monogenic forms represent the infrequent, presenile or early-onset familial Alzheimer's disease (FAD), which is usually characterized by autosomal dominant point mutations of the genes of APP or the presenilin sub-domains of γ-secretase. Both types of mutations facilitate the accumulation of toxic amyloid-i_42, due to abnormal processing or breakdown.

Parkinson's disease (PD) is a progressive neurodegenerative disease characterized by resting tremors, bradykinesia, muscular rigidity, and postural instability. PD typically develops after the age of 60, though 15% of diagnosed patients are under the age of 50. Family history of PD is an etiological factor for 5-10% of patients diagnosed with the disease, yet only 1% of cases have been shown to be clearly familial. It is estimated that 1.5 million Americans are currently living with PD. In an alternative embodiment, PSGF is administered to a subject exhibiting symptoms of PD.

Dementia with Lewy Bodies (DLB) is a progressive brain disease having symptoms that fluctuate between various degrees of manifestation. These symptoms include progressive dementia, Parkinsonian movement difficulties, hallucinations, and increased sensitivity to neuroleptic drugs. As with AD, advanced age is considered to be the greatest risk factor for DLB, with average onset usually between the ages of 50-85. Further, 20% of all dementia cases are caused by DLB and over 50% of PD patients develop “Parkinson's Disease Dementia” (PDD), a type of DLB. It is possible for DLB to occur alone, or in conjunction with other brain abnormalities, including those involved in AD and PD, as mentioned above. In a further embodiment, PSGF is administered to a subject exhibiting symptoms of DLB.

The occurrence of amyloid plaque deposits in the brain may be characteristic of numerous neurodegenerative diseases or other conditions including, but not limited to, Mediterranean fever, Muckle-Wells syndrome, idiopathic myeloma, amyloid polyneuropathy, amyloid cardiomyopathy, systemic neuritic amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, isolated atrial amyloid, β2-microglobulin amyloid in dialysis patients, inclusion body myositis, 2-amyloid deposits in muscle wasting disease, type II diabetes, and combinations thereof. Other embodiments include administering SGF (e.g. PSGF) to subjects exhibiting symptoms of the aforementioned conditions.

Other neurodegenerative conditions associated with oxidative stress include Lou Gehrig's disease and Huntington's disease.

The compounds of the invention may be administered in the form of pharmaceutically acceptable, nontoxic salts, such as acid addition salts. Illustrative of such acid addition salts are hydrochloride, hydrobromide, sulphate, phosphate, fumarate, gluconate, tannate, maleate, acetate, trifluoroacetate, citrate, benzoate, succinate, alginate, pamoate, malate, ascorbate, tartarate, and the like. Particularly preferred antagonists are salts of low solubility, e.g., pamoate salts and the like. These exhibit long duration of activity.

Formulations containing the compounds of the present invention and a suitable carrier can be solid dosage forms which include, but are not limited to, softgels, tablets, capsules, cachets, pellets, pills, powders and granules; topical dosage forms which include, but are not limited to, solutions, powders, fluid emulsions, fluid suspensions, semisolids, ointments, pastes, creams, gels and jellies, and foams; and parenteral dosage forms which include, but are not limited to, solutions, suspensions, emulsions, and dry powder; comprising an effective amount of a polymer or copolymer of the present invention. In some embodiments, a single dose may comprise one or more softgels, tablets, capsules, cachets, pellets, pills, or the like. Specific examples include, for example, a dose comprising 1, 2, 3, or 4 softgels, tablets, capsules, cachets, pellets, pills or the like.

In some embodiments, one or more softgels, tablets, capsules, cachets, pellets, pills, or the like can be taken to achieve the desired dosing. In some embodiments, one or more softgels, tablets, capsules, cachets, pellets, pills, or the like can be taken simultaneously to achieve the desired dosing. In yet another embodiment one or more softgels, tablets, capsules, cachets, pellets, pills, or the like can be taken separately during the course of a specified time period such as for example, a 24 hour period. For example, one or more softgels, tablets, capsules, cachets, pellets, pills, or the like can be taken twice in a 24 hour period to achieve the desired dose. In some embodiments, one or more softgels, tablets, capsules, cachets, pellets, pills, or the like can be taken with a meal. For example one or more softgels, tablets, capsules, cachets, pellets, pills, or the like can be taken with each meal during the course of a 24 hour period to achieve the desired dose.

It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can be administered in the conventional manner by any route where they are active. Administration can be systemic, parenteral, topical, or oral. For example, administration can be, but is not limited to, parenteral, such as subcutaneous, intramuscular, intraperitoneal, intracavity, intrathecal, transdermal, and intravenous. Oral, buccal, or ocular routes, intravaginal, inhalation, depot injections, or implants may also be used to deliver the compounds. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Alternatively, the compounds may be administered as an intranasal spray with an appropriate carrier or by pulmonary inhalation. One suitable route of administration is a depot form formulated from a biodegradable suitable polymer, e.g., poly-D,L-lactide-coglycolide as microcapsules, microgranules or cylindrical implants containing dispersed antagonistic compounds.

Specific modes of administration will depend on the indication. The selection of the specific route of administration and the dose regimen is to be adjusted or titrated by the clinician according to methods known to the clinician in order to obtain the optimal clinical response. The amount of compounds to be administered is that amount which is therapeutically effective. The dosage to be administered will depend on the characteristics of the subject being treated, e.g., the particular animal or human being treated, age, weight, health, types of concurrent treatment, if any, and frequency of treatments, and can be easily determined by one of skill in the art (e.g., by the clinician).

The amount of compound needed depends on the type of pharmaceutical composition and on the mode of administration. In some embodiments, the PSGF compound antagonist administration may be at a dosage of about 0.005 mg/kg/dose to about 100 mg/kg/dose, about 0.005 mg/kg/dose to about 10 mg/kg/dose, about 0.005 mg/kg/dose to about 1 mg/kg/dose, about 0.005 mg/kg/dose to about 0.5 mg/kg/dose, about 0.005 mg/kg/dose to about 0.1 mg/kg/dose, or about 0.005 mg/kg/dose to about 0.05 mg/kg/dose. In cases where human subjects receive solutions of PSGF, administered by i.m. or s.c. injection, or in the form of intranasal spray or pulmonary inhalation, the typical doses are between 2-20 mg/day/patient, given once a day or divided into 2-4 administrations/day. When the GHRH antagonists are administered intravenously to human patients, typical doses are in the range of 8-80 g/kg of body weight/day, divided into 1-4 bolus injections/day or given as a continuous infusion. When depot preparations of the GHRH antagonists are used, e.g. by i.m. injection of pamoate salts or other salts of low solubility, or by i.m. 10 or s.c. administration of microcapsules, microgranules, or implants containing the antagonistic compounds dispersed in a biodegradable polymer, the typical doses are between 1-10 mg antagonist/day/patient.

The SGF compounds can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the SGF compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compounds/compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compounds/compound and a suitable powder base such as lactose or starch.

The compositions of the present invention can also be formulated in rectal compositions such as suppositories or retention enemas, e.g., containing conventional suppository bases such as cocoa butter or other glycerides.

In addition to the formulations described previously, the compositions of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present invention, for example, can be applied to a plaster, or can be applied by transdermal, therapeutic systems that are consequently supplied to the organism.

The SGF compositions can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floe, carboxymethylcellulose, hydroxypropylcellulose, calcium silicate, a metal carbonate, sodium bicarbonate, calcium citrate, or calcium phosphate. [0078] In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

Other suitable excipients for addition to SGF compositions include, for example, those that improve or prolong delivery, bioavailability, absorption or uptake, shelf-life, stability, solubility, efficacy, viscosity, reduce toxicity, improve taste or smell, and combinations thereof. In any of the preferred embodiments the composition of the invention may optionally include, for example, pharmaceutical compounding agents, such as one or more thickening agents such as paraffin oils, esters such as isopropyl myristate, ethanol, silicone oils and vegetable oils, cellulosic thickening agents, ethylcellulose, hydroxypropyl cellulose, hydroxypropyl methylcellulose, povidone, polyacrylic acids such as carbopol, Sepigel® (polyacrylamide/isoparaffin/laureth-7), the Gantrez® series of polymethyl vinyl ether/maleic anhydride copolymers such as the butyl ester of PVM/MA copolymer Gantrez® A-425, and any thickening agent known in the art that has good compatibility with volatile liquids; a preservative, for example, hydroxybenzoate esters; a glycol; water; a surfactant, such as, ethoxylated fatty alcohols, glycerol mono stearate, phosphate esters, and other commonly used emulsifiers and surfactants; a dermal penetration enhancer, for example, octyl salicylate or DMSO; a reducing agent; an emulsifier; an organic solvent, for example, an ether, an ester, an alcohol or an alkane; a triglyceride; a lipid or phospholipid; an oil; a fat; a carbohydrate or saccharide; a protein; a nucleotide; a liposome; a salt or mineral; a plant extract, and the like. In addition it is also contemplated that in any of the preferred embodiments the composition of the invention can be optionally combined with at least one other active agent including another UV absorbing compound, a drug, for example a hormone, an antimicrobial compound, an anti-inflammatory, an antioxidant, and the like.

### Cosmetic Uses and Compositions

Depending on the respective cultural influences, the ideals of beauty are subject to certain transformations. Nevertheless, also today a flawless appearance is of great importance for the majority of the population. In this respect, the condition and the appearance of the skin play a decisive role.

The skin represents a very versatile organ, having a series of essential functions in the human or animal organism, respectively. For instance, on the other hand, the skin provides a barrier, which delimits the body externally and protects it from harmful environmental influences or allows for the exchange with the environment, respectively. On the other hand, the skin has important metabolic functions and is involved, for example, in a significant manner in the defense of pathogens, but also in allergic reactions.

As a consequence of e.g. nicotine and/or alcohol abuse and of the permanent exposition of the skin to influences from the environment such as, for example, UV-radiation, the so-called exogenic skin aging occurs. Moreover, endogenous factors such as the genetic predisposition, additionally cause an aging effect.

One consequence of skin aging is the development of wrinkles due to drying and loss of elasticity in the epidermis. This is accompanied by impaired wound healing and an overall thinner epidermal layer. This causes a stronger visibility of modified blood vessels, in particular in the case of spider veins. A further condition significantly compromising the appearance of the skin is cellulite. Cellulite is not a condition caused by a disease but rather an aesthetic problem, which occurs mostly in women. Therein, the deposition of fat in the subcutis is increased leading, in cases of connective tissue-weakness, to irregular dents on the skin, the so-called orange skin. Also the so-called stretch marks represent an impairment of life quality for many people. Stretch marks are formed by overstretching of the connective tissue in the subcutis, e.g. due to strong weight increase. The overstretching of the connective tissue first leads to blue-reddish stripes; due to the scarring of these tissue ruptures they appear as bright stripes later on, which are distinct from the surrounding skin to a varying degree depending on the pigmentation of the affected skin area.

The cosmetic industry offers various options aiming at counteracting signs of aging in general. The success of the respective products and methods in the long term, however, does often not come up to the expectations of the user. For instance, mainly two methods are used in order to remove spider veins, both of which are connected to side-effects and in both of which frequent relapses occur. Sclerotherapy is an invasive method, in which, besides pain, side-effects such as hematomas and venous thromboses can occur. In laser treatment, which frequently requires multiple sessions, allergic skin reactions and pain can occur as side effects. Recurrence rates are relatively high in both methods.

A cosmetic product according to the disclosure may in particular have one or more of the following effects: (1) healing or recovery of the skin after damage has occurred due to e.g. burns, abrasions, cuts (e.g. cuts generated during shaving), pimples, allergic reactions, poisoning, exposure to radiation (e.g. UV radiation, such as solar radiation, or ionizing radiation, such as radioactive radiation from e.g. radiation therapy or exposure to radioactive nuclear compounds), chemotherapy, contact of the skin with a substance (e.g. chemicals, clothing, soap, make-up, organic matter such as plant fluids or plants), mechanical friction (e.g. shoes, clothing, harnesses, horses), dehydration, bedsores, the application of a tattoo, piercing the skin (e.g. for decorative purposes); (2) recovery and/or maintenance of the homeostasis in the skin during and/or after homeostatic imbalance occurred due to a disease, due to the treatment of a disease, or due to exposure to e.g. heat, cold, drought, radiation, allergenes, poison, substances that are harmful to the skin; (3) improvement of a skin disease or skin disorder, e.g. acne, warts, althlete's foot, Lyme disease, psoriasis, lichen, ichthyosis, keratosis, Darier's disease, pustulosis, herpes zoster, cellulitis, eczema (such as atopic dermatitis), neurodermatitis, herpes, inflammatory skin disorders, children's diseases affecting the skin (such as varicella, rubella, measles); (4) reduction of one or more visible signs of aging of the skin or rejuvenation of the skin, e.g. one or more effects selected from the group of reducing the number and/or the depth of wrinkles, smoothening the skin (including reducing cellulite), restoring the elasticity, reducing the intensity, size and number of age spots and reducing hyperpigmentation. The term “cosmetically effective amount” is an amount of a SGF compound or SGF composition to achieve one or more of the foregoing effects.

According to another embodiment, the present disclosure also relates to a SGF compositions useful for the preparation of a dermatological or cosmetic composition for the treatment of skin. The composition and methods of the present invention are suitable for improving the efficacy of photoprotective cosmetic formulations.

By a “cosmetically effective amount” (e.g., of an antioxidant dermatological or cosmetic composition of the disclosure) is meant a quantity of the composition provided for topical administration and at a particular dosing regimen which is sufficient to achieve a desired appearance, feel, and/or protective effect. For example, an amount that results in the prevention of or a decrease in the symptoms associated with an undesired condition. The amount of the antioxidant composition to be administered to the subject will depend on the type and severity of the condition, the amenability of the condition to respond to the formulated antioxidants, and on the characteristics of the subject and the subject's metabolic ability to respond to the synergistic antioxidant compositions of the disclosure; such characteristics include general health, age, sex, body weight, skin condition, and tolerance to the active agents in the compositions. The skilled practitioner will be able to determine appropriate dosages depending on these and other factors.

In a specific embodiment, disclosed is a method of using the composition in a dermatological or cosmetic application for the treatment of skin, for example, to treat or prevent UV-induced photo-oxidative damage or other damage to skin, cells, or cellular components, for example, lipids, proteins, and nucleic acids of an organism, for example, a human. One aspect of this object comprises administering an effective amount of the composition of the invention in a pharmaceutically acceptable form to a subject in need thereof.

In certain aspects the composition of the invention may be administered together along with any pharmaceutically acceptable carriers, excipients, and/or biologically active or inactive ingredients. Administration of the composition of the invention may be through any suitable dosage form including, for example, creams, lotions, powders, sprays, gels, ointments, a suspension or emulsion, mousses, aerosols, or any one of a variety of transdermal devices for use in the continuous administration of systematically active drugs by absorption through the skin.

### Conjunctive Agents

Compositions for cosmetic or topical administration of the present invention can include other beneficial agents and compounds (conjunctive agents) such as, for example, sun blocking agents, acute or chronic moisturizing agents (including, e.g., humectants, occlusive agents, and agents that affect the natural moisturization mechanisms of the skin), anti-oxidants, sunscreens having UVA and/or UVB protection, emollients, anti-irritants, vitamins, trace metals, anti-microbial agents, botanical extracts, fragrances, dyes and color ingredients, structuring agents, and/or emulsifiers (see U.S. Pat. No. 6,290,938).

1. Sunblock Agents

Sunblock agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (and octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate), anthranilates, ethyl urocanate, homosalate, and Parsol 1789. Non-limiting examples of physical sunblocks include kaolin, talc and metal oxides (e.g., titanium dioxide and zinc oxide). Non-limiting examples of additional sun block agents that are known to those of ordinary skill in the art can be used in the context of the present invention (e.g., International Cosmetic Ingredient Dictionary, 10th edition, 2004, which is incorporated by reference).

2. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil. Non-limiting examples of additional moisturizing agents that are known to those of ordinary skill in the art can be used in the context of the present invention (e.g., International Cosmetic Ingredient Dictionary, 10th edition, 2004, which is incorporated by reference).

3. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite. Non-limiting examples of additional antioxidants that are known to those of ordinary skill in the art can be used in the context of the present invention (e.g., International Cosmetic Ingredient Dictionary, 10th edition, 2004, which is incorporated by reference).

4. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

5. Emulsifiers

In certain aspects of the present invention, the compositions do not include an emulsifier. In other aspects, however, the compositions can include one or more emulsifiers. Emulsifiers can reduce the in interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof. Non-limiting examples of additional emulsifiers that are known to those of ordinary skill in the art can be used in the context of the present invention (e.g., International Cosmetic Ingredient Dictionary, 10th edition, 2004, which is incorporated by reference).

6. Additional Compounds and Agents

Non-limiting examples of additional compounds and agents that can be used with the compositions of the present invention include, vitamins (e.g. D, E, A, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), emollients (i.e. organic esters, fatty acids, lanolin and its derivatives, plant and animal oils and fats, and di- and triglycerides), antimicrobial agents (e.g., triclosan and ethanol), and fragrances (natural and artificial).

### EXAMPLES

In one example, the PSGF varied from C60 to C400, with peak distribution of C120. In a 96 well plate NT-2 cells were cultured and then DCFDA (fluorescence for free radicals) was applied to each well. Cells were exposed to Abeta42, or Abeta42 with different concentrations of PSGF. Using fluorescence microplate reader, the fluorescence intensities of the wells were measured for quantification of free radicals present in wells, and effectiveness of PSGF as an antioxidant. Results indicated that cells exposed to Abeta42 with PSGF had less free radicals present. The fluorescence intensity values for cells exposed to Abeta42 with PSGF were similar to the fluorescence intensity value of cells not exposed to Abeta 42. It can be concluded that PSGF is an effective antioxidant for NT-2 cells exposed to Abeta42. From this study, it is believed that PSGF could be used in Alzhemier's patients to slow down the progression of the disease.

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the preceding definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, any means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A method of treating or preventing an unwanted condition, disease or symptom of a patient associated with oxidative stress comprising administering a therapeutically effective amount or prophylactically effective amount of a small-gap fullerene (SGF) composition to a subject in need.

2. The method of claim 1, wherein said SGF composition comprises SGF compounds ranging from a size of C₆₀ to C₄₀₀.

3. The method of claim 1, wherein the SGF composition comprises SGFs with at least fifty percent being water-soluble SGFs.

4. The method of claim 3, wherein the water-soluble SGFs comprise polyhydroxylate SGFs (PSGFs) or polycarboxylated SGFs.

5. The method of claim 2, wherein said SGF composition comprises a peak distribution of SGF compounds of a size between about C₁₀₀ to C₂₀₀.

6. The method of claim 5, wherein said peak distribution comprises SGF compounds of a size of about C₁₂₀.

7. The method of claim 1, wherein the composition further comprises a pharmaceutically acceptable carrier.

8. The method of claim 1, wherein the oxidative stress is associated with Abeta.

9. The method of claim 1, wherein the unwanted condition is a neurodegenerative disease.

10. The method of claim 9, wherein the neurodegenerative disease is Alzheimer's disease, Parkinson's disease, or Dementia with Lewy Bodies.

11. A method for improving the general condition and appearance of skin, the method comprising administering a cosmetically effective amount of a SGF composition to a patient for cosmetic purposes.

12. The method of claim 11, wherein said SGF composition is administered topically.

13. The method of claim 11, wherein said method diminishes the appearance of wrinkles.

14. The method of claim 11, wherein said SGF composition comprises SGF compounds ranging from a size of C₆₀ to C₄₀₀.

15. The method of claim 11, wherein the SGF composition comprises SGFs with at least fifty percent being water-soluble SGFs.

16. The method of claim 15, wherein the water-soluble SGFs comprise polyhydroxylate SGFs (PSGFs) or polycarboxylated SGFs.

17. The method of claim 11, wherein said SGF composition comprises a peak distribution of SGF compounds of a size between about C₁₀₀ to C₂₀₀.

18. A composition formulated for topical administration, said composition comprising SGFs and cosmetically acceptable carrier.

19. The composition of claim 18, further comprising at least one conjunctive agent.

20. The composition of claim 19, wherein said conjunctive agent comprises sun blocking agents, acute or chronic moisturizing agents, anti-oxidants, sunscreens having UVA and/or UVB protection, emollients, anti-irritants, vitamins, trace metals, anti-microbial agents, botanical extracts, fragrances, dyes and color ingredients, structuring agents, and/or emulsifiers

21. The composition of claim 18, wherein SGFs comprises at least 50 percent PSGFs.
